(19) United States
(12) Patent Application Publication
(10) Pub. No.: US 2006/0095095 A1
(43) Pub. Date: May 4, 2006

(54) Cancer treatment using laser

(75) Inventor: Densen Cao, Sandy, UT (US)

Correspondence Address: Parsons Behle & Latimer, 201 South Main Street, Suite 1800, P.O. Box 45898, Salt Lake City, UT, 84111, US

(73) Assignee: Cao Group, Inc.

(21) Appl. No.: 11/210,276

(22) Filed: Aug. 23, 2005

Related U.S. Application Data
(60) Provisional application No. 60/624,633, filed on Nov. 3, 2004.

(51) Int. Cl. A61N 5/06; A61B 18/18

(52) U.S. Cl. 607/88; 606/15; 606/3

(57) ABSTRACT

A method for killing cancer cells, including placing fiber needles into a human body adjacent cancerous cells, and exposing the cancerous cells to laser light emitted through the fiber needles so that the laser light tends to cause death of the cancer cells.

### DETAILED DESCRIPTION

To date, a laser has not been used to effectively kill cancer cells and treat cancer. Some of the potential advantages of cancer treatment using a laser are as follows:

1). Effective killing of large numbers of cancer cells.

2). Rapid treatment (can be less than 5 minutes).

3). Simple and convenient treatment processes for most of cancers like skin cancer, breast cancer, oral cancer, and etc.

It is now possible to use laser energy to kill cancer cells within the human body as an effective cancer treatment. First, a cancerous tumor is located. Although it would be possible to treat an entire region of the body or an entire body, medical practitioners may prefer to isolate the treatment on a cancerous tumor. The identification methods for location of cancer cells or tumors in the body include but are not limited to laser scanning, magnetic resonance imaging (MRI), x-ray imaging, palpation and others.

After the location of cancer cells is determined, laser energy of a desired wavelength, intensity, duration and modulation is delivered to the cancer cells. Delivery may be direct laser application to the affected region of the body. Alternatively, a fiber needle or fiber can be used to deliver laser light/laser energy to cancer cells. The fiber needle assembly to delivery laser energy to cancer cells can be one or multiple fibers depending on the size of the tumor. Multiple fiber needles can be inserted inside the body in different directions so that the cancer tumor can be surrounded or covered completely by laser energy coming at the tumor from different directions.

Lasers which emits laser energy used to kill cancer can be solid state lasers, gas lasers, semiconductor lasers and others. An example of wavelengths of laser light effective in killing human cancer cells are from 200 nm to 5000 nm. Wavelengths outside this range may also be used. The energy level of a laser used to kill cancer cells may be from 0.1 watt to 15 watts, or it may be a level outside that range. An example treatment time for exposing cancer cells in a human body to laser energy can be from less than 1 minute to more than 1 hour, or some time frame outside that range. The laser energy applied to the cancer may also be modulated. Laser energy may be applied to cancer cells by continuous wave (constant level), pulsing (on/off), ramping (from low to high power levels, or from high to low power levels), or other waveform (such as sine wave, square wave, triangular wave, etc.). Modulation of laser energy may be achieved by modulating power to the laser light source, or by blocking or reducing light output from the laser light source according to a desired modulation pattern. In one test, a laser light source emitting laser light at about 810 nm at a power level of 5 watts was used to expose human cancer cells to laser light for about 5 minutes, resulting in death of all of substantially all of the cancer cells so exposed.

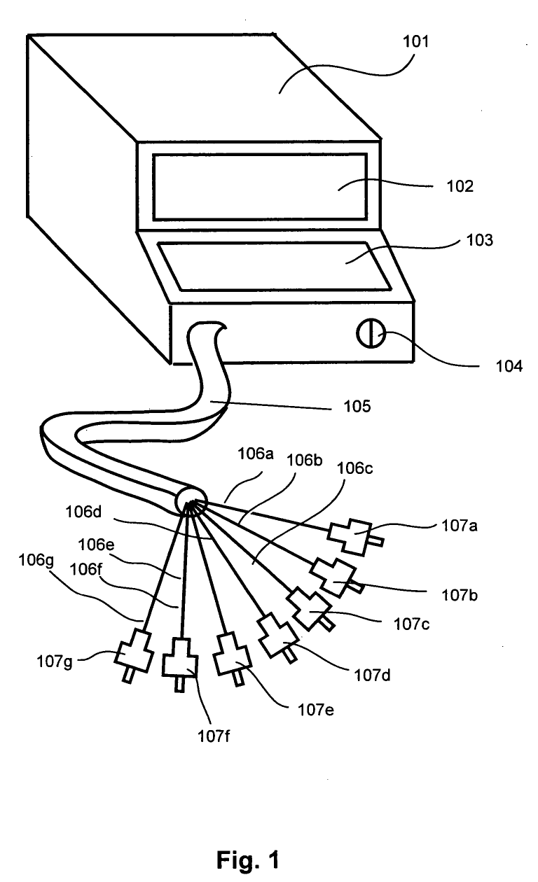

FIG. 1 depicts an example laser system 101 that can be used for cancer treatment. The laser system 101 contains a laser light source, control circuits, and other managing/control components, power supply and circuitry. A display panel 102 displays all laser and treatment information. A control panel 103 has buttons or switches to control the laser's operation. A key switch 104 may be used to control the main electrical on/off for safety reasons. A fiber bundle cable 105 may be used to transport light out of the main laser module to some remote location for therapeutic use. The fiber bundle may be broken down into contains numerous individual fiber 106a through 106g. Each fiber may have an end connector 107a through 107g respectively, to facilitate the transmission of laser energy from the laser system to a delivery device for delivering laser energy to cancer cells.

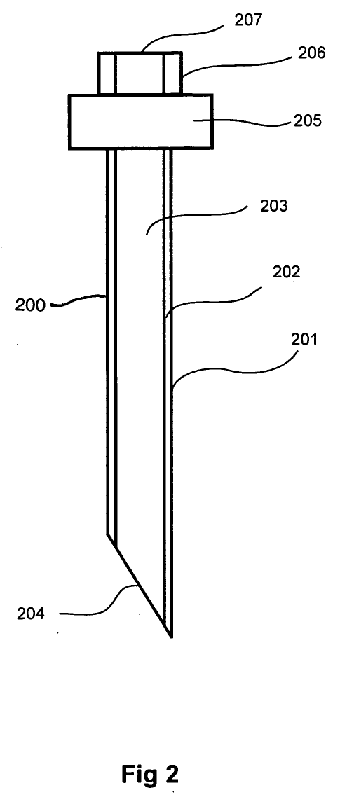

FIG. 2 depicts an example fiber needle 200 that can be used to deliver the laser energy to cancer cells. The fiber needle may include a rigid housing (such as metal or plastic) with a stem 201, a channel 202, and a fiber 203 inside the channel. The end of the needle may have a sharp point and an angled surface 204. The fiber is polished to the same angle as metal housing to create a sharp point for insertion. Laser energy is delivered to through fiber. The top side of needle has fiber connector 206 and abutment 205 so that the needle can connect to the fiber with the connector from the laser unit. The top side of needle is polished surface 207 for connection to the connector from individual fiber of the fiber bundle mentioned above. The sharp fiber needle may be inserted into the body in any location where cancerous cells are believed to be located in order to deliver laser light directly to those cells.

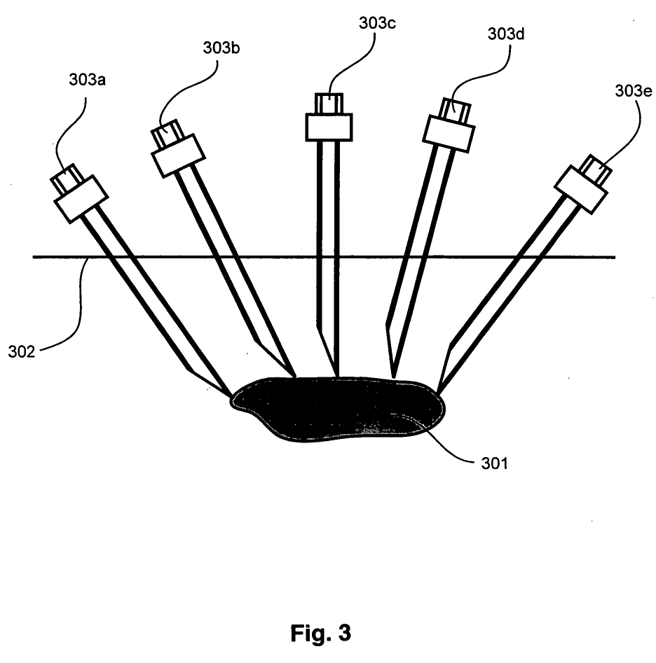

FIG. 3 depicts an example of using multiple fiber needles to deliver laser energy to cancer cells. If desired, laser energy may be delivered to cancer cells at one or more points such as depicted, or it may be delivered in a footprint covering a larger area if desired. A cancer tumor 301 in a human body below the skin surface 302 is located, and fiber needles 303a, 303b, 303c are inserted into the human body and pointed toward the tumor. It is possible to deliver the laser energy from outside the body without a needle invading the body, but it may be desirable to insert needles into unaffected tissue so that laser energy may be delivered directly to the tumor. The fiber needles may surround or partially surround the cancer tumor. The number of fiber needles to be used treatment depends on the side and location of cancer tumor. The depth of the needle insertion depends on the location of cancer tumor. The length or height of fiber needle can be different based on the requirement in different treatment situations.

If desired, a dye or ink may be injected into the tumor prior to laser treatment. Dye or ink can be used to facilitate absorption of the laser light, creating heat and killing the cancer cells. Alternatively, the laser light may be selected to be of a wavelength that cancer cells tend to absorb but which tends to pass through healthy cells harmlessly.

While compositions and methods have been described and illustrated in conjunction with a number of specific ingredients, materials and configurations herein, those skilled in the art will appreciate that variations and modifications may be made without departing from the principles herein illustrated, described, and claimed. The present invention, as defined by the appended claims, may be embodied in other specific forms without departing from its spirit or essential characteristics. The configurations of snacks described herein are to be considered in all respects as only illustrative, and not restrictive. All changes which come within the meaning and range of equivalency of the claims are to be embraced within their scope.

I claim:

1. A method for treating a cancerous tumor within a human body comprising the steps of:
locating a region within a human body that contains cancer cells,
obtaining access to a laser system having multiple fibers through which laser light may be emitted,
attaching fiber needles to at least a plurality of said fibers so that laser light from said laser system may pass through said fibers and thence through said fiber needles,
inserting said fiber needles into the human body so that the ends of said fiber needles are in close proximity to the cancer cells and so that the fiber needles tend to point in the direction of the cancer cells,
causing emission of laser light from the laser system, through the fibers, through the fiber needles and thence to the cancer cells,
continuing said emission of laser light for a medically effective duration in order to kill at least some of the cancer cells.

2. A method as recited in claim 1 further comprising the step of placing a dye in the region of the cancer cells in order to enhance absorption of laser light by the cancer cells.

3. A method as recited in claim 1 further comprising the step of selecting said laser light of a wavelength which tends to be absorbed by cancer cells but which tends to pass harmlessly through healthy cells.

4. A method as recited in claim 1 wherein said fiber needle has a sharp tip.

5. A method as recited in claim 1 wherein said fiber needle includes an exterior metal sheath encasing a fiber capable of transporting laser light.

6. A method as recited in claim 5 wherein said fiber and said metal sheath terminate together at a sharp tip.

7. A method as recited in claim 1 wherein said laser is selected from the group consisting of semiconductor lasers, solid state lasers, and gas lasers.

8. A method as recited in claim 1 wherein said laser emits light of a wavelength within the range of from 200 nm to 5000 nm.

9. A method as recited in claim 1 wherein said laser emits light of a power level in the range of from 0.1 watt to 15 watts.

10. A method as recited in claim 1 wherein said cancer cells are exposed to said laser light for a time duration that is within the range of from 1 minute to 1 hour.

11. A method as recited in claim 1 wherein said laser light is maintained in continuous wave format as it is exposed to said cancer cells.

12. A method as recited in claim 1 wherein said laser light is modulated as it is exposed to said cancer cells.

13. A method as recited in claim 12 wherein said modulation is selected from the group consisting of puling, ramping, sine waveforms, square waves and triangular waves.

14. A method as recited in claim 1 wherein said laser light has a wavelength of about 810 nm.
